(19) United States
(12) Patent Application Publication
(10) Pub. No.: US 2013/0046250 A1
(43) Pub. Date: Feb. 21, 2013

(54) Drive Mechanism for Drug Delivery Devices

(71) Applicant: Andreas Bode, Frankfurt am Main (DE)

(72) Inventor: Andreas Bode, Frankfurt am Main (DE)

(73) Assignee: SANOFI-AVENTIS DEUTSCHLAND GMBH, Frankfurt am Main (DE)

(21) Appl. No.: 13/583,753

(22) PCT Filed: Dec. 1, 2010
(86) PCT No.: PCT/EP2010/068596

§ 371 Date: Nov. 1, 2012

(30) Foreign Application Priority Data
Dec. 2, 2009 (EP) 09177685.6

(51) Int. Cl. A61M 5/315

(52) U.S. Cl. 604/228

(57) ABSTRACT

The present invention relates to an ergonomic drive mechanism for a drug delivery device comprising:
a housing (36),
an axially displaceable piston rod (12) to act on a piston (18) of a cartridge (16) containing a medicinal product to be dispensed,
an actuating member (26) operably engageable with the piston rod (12) and being slidably disposed relative to the housing (36) between a distal and a proximal stop position along a displacement path,
   wherein the actuating member's displacement path is inclined with respect to the longitudinal extension of the piston rod (12).

### FIELD OF THE INVENTION

This invention relates to a drive mechanism for a drug delivery device that allows a user to select single or multiple doses of an injectable medicinal product and to dispense the set dosage of the product and to apply said product to a patient, preferably by injection. In particular, the present invention relates to such devices, which are handled by the patients themselves.

### BACKGROUND AND PRIOR ART

Drug delivery devices allowing for multiple dosing of a required dosage of a liquid medicinal product, such as a liquid drug, and further providing administration of the liquid to a patient, are as such well-known in the art. Generally, such devices have substantially the same purpose as that of an ordinary syringe.

Drug delivery devices of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose. In order to meet these requirements, the number of parts and steps required to assemble the device and an overall number of material types the device is made from have to be kept to a minimum.

With some of the known pen-type injectors, for setting or preparing of a dose in a first step, a push button or a respective push-pull rod is at least partially displaced in a proximal direction. Starting from a proximal stop position, a pre-defined dose of the medicinal fluid can be dispensed and administered by a user inducing or initiating exertion of a distally directed pressure to said push button or push-pull rod, respectively.

With these known devices and drive mechanisms, a user has to exert a certain stress or pressure to the push button or to a respective push-pull rod at least for initiating or triggering a dose dispensing action. Depending on the overall geometry and functionality, the push button or the respective push-pull rod is to be typically depressed and displaced by a user's thumb. The displacement path of said push button or push-pull rod typically increases with the size of the dose to be set or dispensed. In particular, for users with comparatively small hands or fingers, the device handling may become quite laborious and awkward. Furthermore, the displacement path of push buttons of conventional devices is substantially linear and straight and does not ideally match with physiologically governed limitations of the respective user.

### OBJECT OF THE INVENTION

It is therefore an object of the present invention to provide an improved and particularly user-friendly drive mechanism for a drug delivery device, which provides easy and smooth device handling, in particular for dispensing of large doses. The invention also focuses on a robust and reliable drive mechanism, which is inexpensive in production and which requires a limited number of components. The invention also focuses on an ergonomically optimized design particularly in terms of device handling.

### SUMMARY OF THE INVENTION

In a first aspect, the invention provides a drive mechanism for a drug delivery device adapted for dispensing of a single or multiple doses of a medicinal product, typically a medicinal fluid, such as a fluid drug like insulin or heparin. The drive mechanism comprises a housing and an axially displaceable piston rod, which is adapted to act on a piston of a cartridge containing the medicinal product to be dispensed. The piston rod may be disposed along the main longitudinal axis of the cartridge. The piston rod may be rigid. The piston rod is adapted to abut with a proximal end face of the slidably disposed piston. Upon exertion of distally directed thrust or pressure, the piston rod drives and pushes the cartridge's piston in distal direction which in turn leads to the intended dispensing of the pre-defined dose of the medicinal product.

The drive mechanism further comprises an actuating member engageable with the piston rod and being slidably disposed relative to the housing between a distal and a proximal stop position along a displacement path. Distal and proximal stop position may vary. The proximal stop position is typically correlated with the size of a selected dose and the actuating member is typically designed as a push-pull element. It is even conceivable, that the actuating member is rotatably engaged or coupled with the housing of the drive mechanism.

The actuating member passes along a displacement path when displaced from proximal to distal stop position, e.g. during dose dispensing or when displaced from distal to proximal stop position, e.g. during dose setting. According to an embodiment, this displacement path is inclined or slanted with respect to the longitudinal extension of the piston rod.

The bearing and/or mounting of the actuating member in the drive mechanism's housing is such, that the trajectory described by the actuating member during its dose setting- or dose dispending displacement is at least non-parallel to the longitudinal access defined by the shape and geometry of the piston rod and/or the cartridge that contains the medicinal product.

Preferably, inclination of the actuating member's displacement path is such, that the actuating member can be displaced in an ergonomic way by means of a user's thumb. By its tilted or inclined orientation, the actuating member and its displacement path accommodate physiological demands of a user and match with ergonomic requirements.

In a typical device handling situation, the actuating member's displacement path is inclined towards the thumb of a user. In this way, a user can easily and ergonomically cope with situations, in which the actuating member has to be displaced along a comparatively large displacement path. By means of the inclination of the actuating member's displacement path, the overall device handling becomes user friendlier and safer. Moreover, the inclination of the displacement path allows for a variety of different housing designs that may even deviate from the conventional cylindrical shape.

Such a physiologically and ergonomically adapted device design and/or device handling is particularly beneficial for users being physically infirm and which may additionally suffer sequalae or late complications.

According to a preferred aspect, the displacement path is curved or arcuate. Such a curved shape or trajectory is particularly beneficial for a thumb-induced or thumb-governed actuation because it resembles a curved or arcuate trajectory of the free end of a user's thumb. By providing a curved or arcuate displacement path, the drive mechanism and the drug delivery device match with the physiological properties of a user's hand and a thumb may follow its natural and physiologically-given trajectory, especially during dose dispensing.

In another preferred embodiment, the actuating member itself comprises a curved or arcuate shape. The displacement path described by e.g. a proximal end section of the actuating member may then substantially overlap with the actuating member's shape. However, it is also conceivable, that the actuating member is pivot-mounted or translationally mounted in the housing and/or with respect to the piston rod.

In a further embodiment, the actuating member intersects a sidewall section or a proximal end section of the housing. Hence, the housing comprises a corresponding through opening either at a sidewall section or at its proximal end section to guide the actuating member there through. In this embodiment, it is beneficial, when the actuating member is translationally guided by said through opening.

By having a sideward intersection of actuating member and housing, a particularly ergonomic design of a respective housing can be provided. Also, the overall shape of the housing does no longer have to be of substantially cylindrical shape but can be freely designed, e.g. in a droplet form resembling and matching with an inner contour or surface of a closed hand.

According to another preferred embodiment, the actuating member and the piston rod are releasably engaged. The actuating member and the piston rod may be releasably engaged by means of at least one gear wheel. The gear wheel may mesh with the piston rod and/or the actuating member, in particular with toothed surfaces of the piston rod and/or the actuating member. In this way, a kind of rack and pinion drive can be realized, that translates a push-pull motion of the actuating member into a respective rotational motion of the at least one gear wheel. The rotational motion of the gear wheel can then in turn be used to drive the piston rod in distal direction for dispensing of the set dose.

Preferably, the drive mechanism comprises a first and a second gear wheel, each of which meshing with a corresponding toothed surface of the actuating member and/or the piston rod. First and second gear wheels are preferably operably engaged and/or releasably coupled to each other. Typically, first and second gear wheels are arranged on a common rotatable shaft.

In this way, by having two interleaved rack and pinion drives, a translational movement of the actuating member can be transferred into a rotational movement of the shaft, which in turn is transferred to a respective translational and distally directed movement of the piston rod. Typically, the actuating member at least in sections comprises a toothed surface meshing with a first gear wheel and the piston rod comprises a toothed surface that meshes with a second gear wheel, wherein first and second gear wheels are operably engaged by means of the common rotatable shaft.

Depending on the overall dimensions and the size of the gearing, first and second gear wheels may both be rigidly connected to the common shaft. Alternatively, it is conceivable, that a transmission gear is implemented, e.g. by making use of differently toothed gear wheels. First and second gear wheels may be optionally coupled and mutually engaged by means of a separate transmission gear. Also, a releasable coupling of first and second gear wheels among themselves as well as with respect to geared surfaces of actuating member and/or piston rod might be provided.

In another preferred embodiment, the at least one gear wheel is unidirectionally engaged with the shaft and/or with the second gear wheel by means of a ratchet-like coupling. In this way, a unidirectional, e.g. distally directed displacement of the piston rod can be implemented. Hence, for setting of a dose, the actuating member with its toothed surface can be pulled out and brought into its proximal stop position. This dose setting motion of the actuating member may be accompanied by a respective rotation of the first gear wheel. Due to the ratchet-like coupling, the rotation of the first gear wheel is neither transferred to the shaft nor to the second gear wheel. Consequently, the piston rod remains fixed, preferably in an abutment position with the cartridge's piston.

As soon as the actuating member is displaced in an opposite, distal direction, by means of the ratchet-like coupling, also the shaft and/or the second gear wheel is set in respective motion leading to a respective dose-dispensing distal displacement of the piston rod.

According to another preferred embodiment, the shaft itself or at least one of said gear wheels is slidably disposed in the housing along the shaft's longitudinal direction for mutually engaging and/or disengaging of piston rod and actuating member. Typically, the shaft is oriented perpendicular to a plane defined by the extension of the piston rod and the extension of the actuating member. By displacing the shaft and/or any one of the interconnected gear wheels in a direction substantially perpendicular to said plane, a mechanical coupling of piston rod and actuating member can be selectively disengaged. Such disengagement is particularly applicable for setting of a dose by displacing of the actuating member in proximal direction. When the shaft and/or any one of first and second gear wheels are slidably disposed in the housing, first and/or second gear wheels may be permanently connected and coupled to the shaft.

In a further preferred embodiment, the at least one gear wheel and/or the shaft is engaged with a display member, which is at least adapted to indicate an engagement of piston rod and actuating member. In this way, the user can be informed on the actual configuration of the drive mechanism. Furthermore, the display member may also be adapted to indicate an actual position of the piston rod and/or an actual position of the actuating member. In this way, the user can be informed on the filling level of the cartridge and/or the size of the set dose.

The display member might be displaceably disposed in the housing and may protrude from the housing with a significant colour, e.g. for informing the user, that a particular dose is set and/or that the device is prepared and ready for dose dispensing. Typically, in dose dispensing configuration, actuating member and piston rod are operably engaged.

According to another beneficial aspect, the actuating member at least comprises a flexibly deformable section. The flexibly deformable section may be the distal end section of the actuating member. The flexibly deformable section may be adapted to be bended by an inner sidewall of the housing. In particular, the flexible distal end section is to be bended in response to an abutment with an inside surface of the housing in the course of a dose dispensing action, hence during a distally directed displacement of the actuating member. The distal end section may be elastically flexibly deformable. The distal end section of the actuating member may take its original, e.g. unbended, shape when not mechanically cooperating with the inside surface of the housing, for example. Flexible deformation and bending of the actuating member allows for a compact design of the housing as well as for the realization of large displacement paths the actuating member is to be displaced along. The actuating member comprises a proximal end section. The proximal end section of the actuating member may be less flexibly deformable than the distal end section of the actuating member.

In a further preferred embodiment, the arcuate shaped actuating member extends between a guiding element and the at least one gear wheel. Here, the at least one gear wheel engages with an inside directed toothed surface of the actuating element while the guiding element engages with an outside directed surface of the actuating element. Preferably, guiding element and outside directed surface of the actuating element are substantially plane in order to provide a smooth and easy mutual guiding of actuating element and guiding element. In particular, in connection with a flexibly deformable distal end section, the gap between guiding element and first gear wheel substantially matches the thickness of the toothed flexible end portion of the actuating element. In this way, the actuating element can be positively guided by the gear wheel and the guiding element.

In a further preferred embodiment, the guiding element is displaceably arranged in a through opening of the housing. The through opening may be a breakthrough in the housing, for example. The through opening may comprise a size suited to receive the guiding element, in particular to enable at least partial insertion of the guiding element into the housing and, thus, mechanical cooperation of the guiding element and the actuating member. The through opening may be adapted and arranged to enable movement of the guiding element in the radial direction with respect to the housing. The guiding element may be externally actuatable. The through opening may be configured such that an outer or actuating part of the guiding element may be prevented from being inserted into the housing. Said through opening is arranged preferably opposite to the through opening though which the curved or arc-shaped actuating member protrudes from the housing. Preferably, the guiding element may provide a kind of clutch function. When depressed by the user, the gap between guiding element and gear wheel is narrowed leading to a positive fitting and engagement of the rack and pinion drive of actuating member and gear wheel. If released, the guiding element may be displaced in an outward direction under the effect of a reset spring force, which may be provided by a separate spring element or even by the flexibly bended actuating member itself.

According to an embodiment, the guiding element is depressible against a reset spring force provided by the actuating member. The actuating member, in particular the flexibly deformable distal end section of the actuating member, may provide a radially outwardly directed elastic force, i.e. the reset spring force. Said force may help preventing mechanical cooperation of the actuating member and the gear wheel, e.g. for resetting the device or for setting a dose. In particular, due to the reset spring force, the flexibly deformable distal end section of the actuating member automatically bents radially outwardly. The radially outwardly directed force is counteracted when depressing the guiding element, in particular when moving the guiding element radially inwardly with respect to the housing. Accordingly, when depressing the guiding element, the actuating member is bent radially inwardly by mechanical cooperation with the guiding element, the actuating member thus being brought into engagement with the gear wheel.

In the reset configuration, in which the guiding element is shifted outwardly, actuating element and gear wheel become disengaged, e.g. for the purpose of setting of a dose. As soon as the guiding element is depressed, e.g. by any one of the residual fingers of a user's hand, an engagement of the actuating member and the gear wheel can be established, which is required for dose dispensing.

According to another independent aspect, the invention also provides a drug delivery device for dispensing of a medicinal product, such as heparin or insulin, wherein the device comprises a drive mechanism, preferably a drive mechanism as presently described.

It will be apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

Without limitation, the present invention will be explained in greater detail below in connection with preferred embodiments and with reference to the drawings in which:

FIG. 1 schematically illustrates a rack and pinion coupling of actuating member and piston rod,

FIG. 2 schematically illustrates a drive mechanism as implemented in a drug delivery device during dose dispensing to be conducted by a user,

FIG. 3 exemplary illustrates a first embodiment of a display member and

FIG. 4 shows another embodiment of a display member for indicating the filling level of the cartridge and/or the size of the set dose.

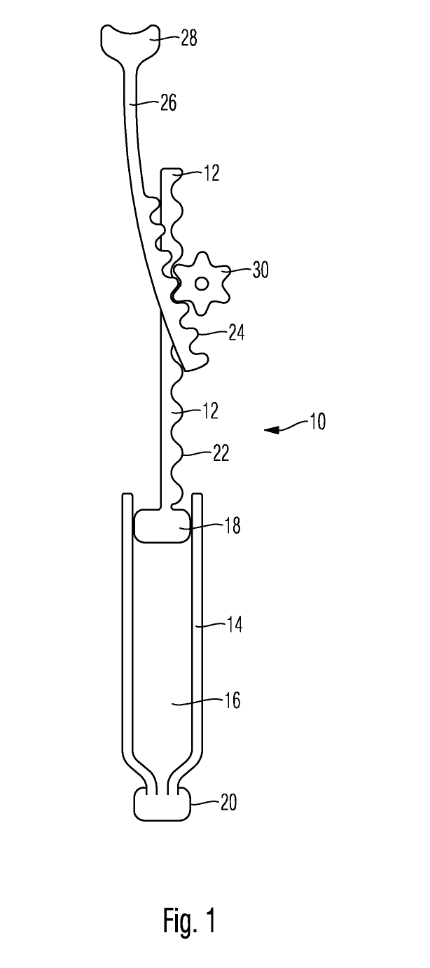

The drive mechanism 10 as illustrated in FIG. 1 comprises an actuating member 26 of curved or arcuate shape. The actuating member 26 comprises a distal end section. The actuating member 26 comprises a proximal end section. The distal end section is flexibly deformable, preferably elastically flexibly deformable. The proximal end section is less flexibly deformable than the distal end section of the actuating member 26. At its upper and proximal end section, the rod-like actuating member 26 comprises a flange-like radially widened push-button 28. At its lower and distal end section, the actuating rod 26 comprises a toothed surface 24 that meshes with a gear wheel 30. The gear wheel 30 is rotatably supported by a not further illustrated shaft. In a similar way, a not further illustrated second gear wheel meshes with a corresponding toothing 22 of a piston rod 12. First and second gear wheels 30 are either releasably or permanently coupled with respect to each other and can be of varying thread pitches. Having such an interleaved two-fold rack and pinion drive, an arc shaped or circular-segment-like displacement path of the actuating rod 26 can be provided.

The arcuate displacement path is particularly beneficial with respect to an ergonomic device handling. By means of the two-fold rack and pinion drive, an arcuate shaped actuating motion of the actuating rod 26 can be transferred to a unidirectional linear translational displacement of the piston rod 12.

The piston rod 12 is rigid. The piston rod 12 is disposed along the main longitudinal axis of the cartridge 14. The piston rod 12 is operably engaged with a piston 18 of a cartridge 14 that contains the medicinal fluid 16 to be dispensed via an outlet portion 20 of the cartridge 14. The distally directed outlet 20 of the cartridge 14 is to be connected with a not further illustrated needle or cannula in a fluid-transferring way.

The term “medicinal fluid”, as used herein, preferably means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38[Asp28] Exendin-4(1-39)-NH2,

### H-Asn-(Glu)5des Pro36, Pro37, Pro38[Asp28] Exendin-4(1-39)-NH2,

### des Pro36, Pro37, Pro38[Asp28] Exendin-4(1-39)-(Lys)6-NH2,

### H-(Lys)6-des Pro36, Pro37, Pro38[Asp28] Exendin-4(1-39)-(Lys)6-NH2,

### H-Asn-(Glu)5-des Pro36, Pro37, Pro38[Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38[Trp(O2)25] Exendin-4(1-39)-NH2,

### H-(Lys)6-des Pro36, Pro37, Pro38[Trp(O2)25, Asp28] Exendin-4(1-39)-NH2,

### H-Asn-(Glu)5-des Pro36, Pro37, Pro38[Trp(O2)25, Asp28] Exendin-4(1-39)-NH2,

### des Pro36, Pro37, Pro38[Trp(O2)25, Asp28] Exendin-4(1-39)-(Lys)6-NH2,

### H-(Lys)6-des Pro36, Pro37, Pro38[Trp(O2)25, Asp28] Exendin-4(1-39)-(Lys)6-NH2,

### H-Asn-(Glu)5-des Pro36, Pro37, Pro38[Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38[Met(O)14, Asp28] Exendin-4(1-39)-NH2,

### H-Asn-(Glu)5-des Pro36, Pro37, Pro38[Met(O)14, Asp28] Exendin-4(1-39)-NH2,

### des Pro36, Pro37, Pro38[Met(O)14, Asp28] Exendin-4(1-39)-(Lys)6-NH2,

### H-(Lys)6-des Pro36, Pro37, Pro38[Met(O)14, Asp28] Exendin-4(1-39)-(Lys)6-NH2,

### H-Asn-(Glu)5 des Pro36, Pro37, Pro38[Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38[Met(O)14, Trp(O2)25] Exendin-4(1-39)-NH2,

### H-(Lys)6-des Pro36, Pro37, Pro38[Met(O)14, Asp28] Exendin-4(1-39)-NH2,

### H-Asn-(Glu)5-des Pro36, Pro37, Pro38[Met(O)14, Trp(O2)25, Asp28] Exendin-4(1-39)-NH2,

### des Pro36, Pro37, Pro38[Met(O)14, Trp(O2)25, Asp28] Exendin-4(1-39)-(Lys)6-NH2,

### H-(Lys)6-des Pro36, Pro37, Pro38[Met(O)14, Trp(O2)25, Asp28] Exendin-4(S1-39)-(Lys)6—NH2,

### H-Asn-(Glu)5-des Pro36, Pro37, Pro38[Met(O)14, Trp(O2)25, Asp28] Exendin-4(1-39)-(Lys)6—NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

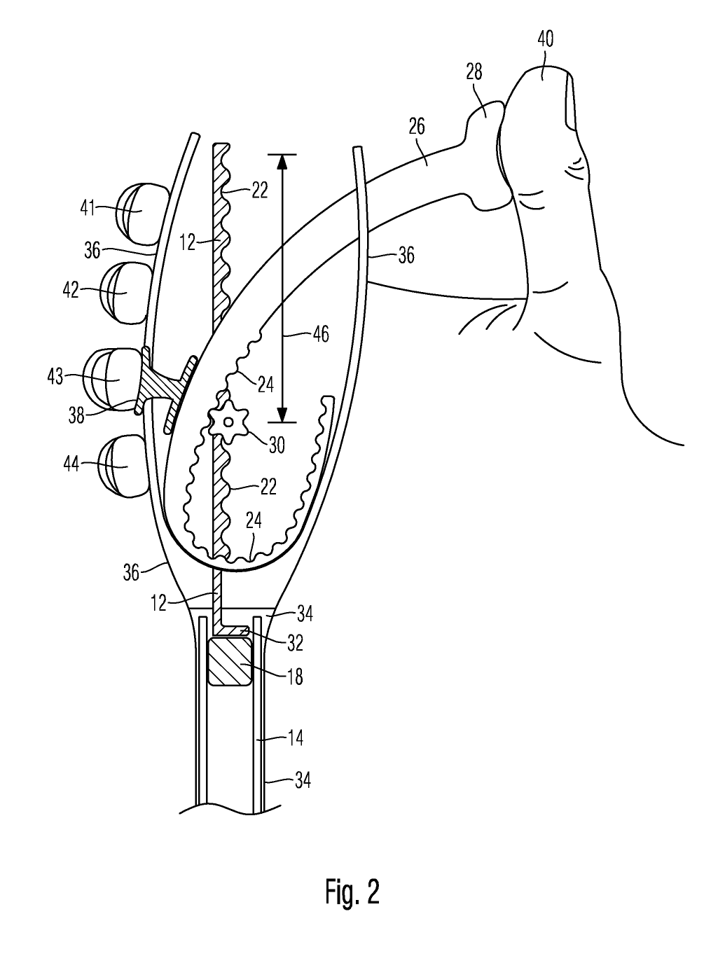

Due to the rack and pinion drive based mechanical coupling of actuating rod 26 and piston rod 12, the actuating rod 26 can be almost arbitrarily oriented inside the housing 36 of the drug delivery device. As illustrated in FIG. 2, the actuating rod 26 is inclined towards the right hand side, namely towards a thumb 40 of a user's hand. In this way, the device itself and its handling can be adapted to the physiological properties of the user. The displacement path described by the actuating rod 26 resembles the natural and physiologically specified circular-segment-like movement of a user's thumb 40.

As can further be seen in FIG. 2, the actuating rod 26 intersects a sidewall portion of the housing 36. The housing 36 itself also comprises a particularly ergonomic and convex shape. The housing 36 can thus be easily gripped by the palm of a hand and its residual fingers 41, 42, 43, 44.

In the illustrated embodiment, the ring finger 43 rests against a guiding element 38. The guiding element 38 is displaceably, in particular radially displaceably, arranged in a through opening of the housing 36. The through opening may be a breakthrough in the housing 36, for example. The guiding element 38 thereby not only serves to guide the flexible deformable lower part or distal end section of the actuating rod 26. Since it is displaceably mounted in the housing 36, it may further provide a clutch functionality. By depressing the guiding element 38, a radially inwardly directed force is transferred to the actuating rod 26 by the guiding element 38. The radially inwardly directed force counteracts a radially outwardly directed force, e.g. an elastic reset spring force, provided by actuating rod 26, in particular by the flexibly deformable distal end section of the actuating rod 26. Thereby, the flexibly deformable distal end section of the actuating rod 26 is bent or moved radially inwardly. Accordingly, by depressing the guiding element 38, the flexibly deformable actuating rod 26 is brought into engagement with the gear wheel 30 and the actuating rod 26 becomes operably engaged with the piston rod 12. A distally, hence downwardly directed displacement of the actuating rod 26 towards its distal stop position is then transferred to the piston rod 12, which by means of its pressure piece 32 transfers a respective distally directed displacement to the cartridge's 14 piston 18.

When releasing the guiding element 38, no force is available any more to counteract the radially outwardly directed elastic force provided by actuating rod 26. Thus, by releasing the guiding element 38, the actuating rod 26 and the gear wheel 30 become disengaged. In particular, the actuating rod 26 and the gear wheel 30 become disengaged as the flexibly deformable distal end section of the actuating rod 26 bents or moves radially outwardly driven by the radially outwardly directed elastic force provided by the distal end section of the actuating rod 26. Releasing of actuation rod 26 and gear wheel 30 might be governed by the elasticity and pre-tension of the actuating rod 26 itself. If released, the actuating rod 26 will tend to displace the guiding element 38 in an outward direction.

As can be further seen in FIG. 2 the lower and bended end section of the actuating rod 26 bends in an almost spiral-like way along an inner surface of the housing 36. Due to the elasticity and bending of the actuation rod 26, the overall housing 36 can be designed comparatively compact. Furthermore, the distance 46 indicates the length of the cartridge 14 or the maximum path length, the cartridge's 14 piston 18 can be displaced. As further illustrated, the cartridge 14 is disposed inside a cartridge holder 34 which in turn is permanently and/or releasably interconnected with the housing 36 of the drug delivery device.

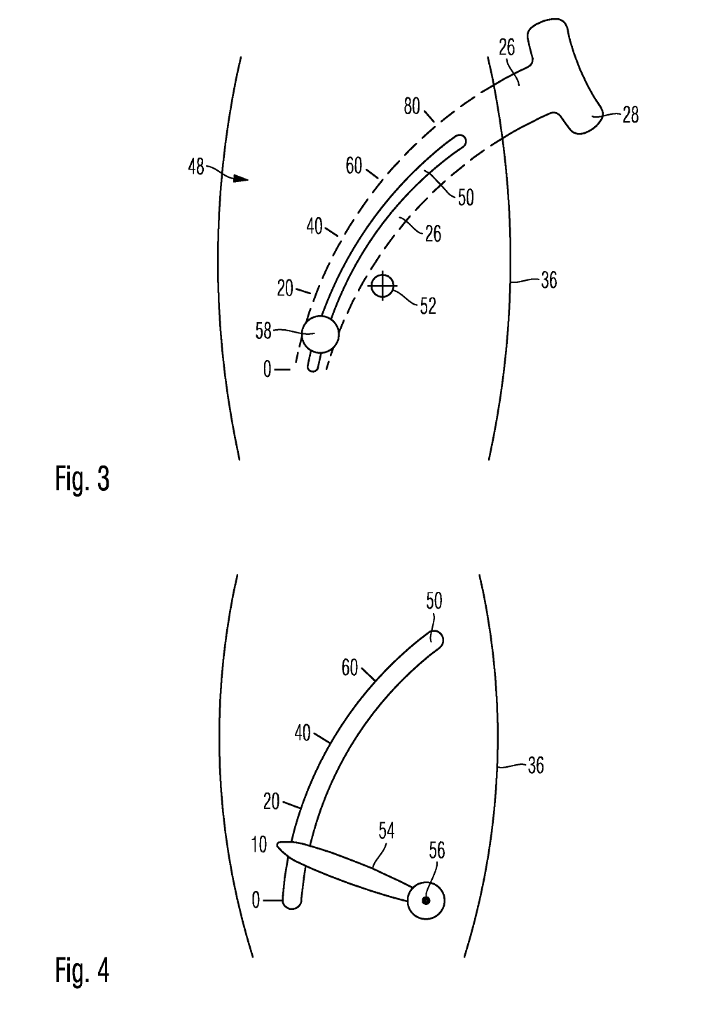

The rack and pinion coupling of the actuation rod 26 and the at least one gear wheel 30 can be modified in different ways. It is possible for instance, that the shaft and/or the gear wheel 30 are displaceably arranged in the housing 36 along the gear's rotation axes. By shifting at least one gear wheel 30 in this direction, piston rod 12 and actuation rod 26 can be selectively engaged and disengaged, depending on whether a dose is to be set or to be dispensed. A displacement of the shaft or gears 30 can further be coupled with a display member 52 as illustrated in FIG. 3. The display member 52 may have a significant colour and may protrude from the outside of the housing 36. In this way, the display member 52 indicates, whether the drug delivery device is in dose setting or dose dispensing mode. It may further indicate whether the actuating member 26 and the piston rod 12 are operably engaged or not.

The housing as illustrated in FIG. 3 further comprises an arcuate slit 50 provided with a scale 48. The slit 50 serves as a guiding means for an indicator 58, which is directly or indirectly coupled to the actuating rod 26. By pulling the actuating rod 26 in proximal direction, the respective position of the indicator 58 is informative on the size of the set dose. In another embodiment sliding the indicator 58 can be used for setting a dose. Also, the slit 50 may serve as a means for guiding and securing the actuating rod 26 with respect to the housing 36.

In FIG. 4, another embodiment of a display member 54 is illustrated. Here, the display member 54 comprises a pivot mounted indicator 54 supported by an axis 56. The indicator 54 is preferably coupled to the actuating rod 26 or to a gear wheel 30 meshing with said rod 26. Alternatively, the indicator 54 may also be operably coupled to the piston rod 12 or to a respective gear wheel that meshes with the piston rod 12. In such a configuration, the orientation and position of the indicator 54 is informative of the filling level of the cartridge 14 and/or the size of the set dose.

### REFERENCE NUMERALS

10 drive mechanism
12 piston rod
14 cartridge
16 liquid drug
18 piston
20 outlet portion
22 toothing
24 toothing
26 actuating rod
28 push-button
30 gear wheel
32 pressure piece
34 cartridge holder
36 housing
38 guiding element
40 thumb
41 finger
42 finger
43 finger
44 finger
46 length
48 scale
50 slit
52 display member
54 indicator
56 axis
58 indicator

1. A drive mechanism for a drug delivery device, comprising:
a housing,
an axially displaceable piston rod to act on a piston of a cartridge containing a medicinal product to be dispensed,
an actuating member operably engageable with the piston rod and being slidably disposed relative to the housing (36) between a distal and a proximal stop position along a displacement path,
wherein the actuating member's displacement path is inclined with respect to the longitudinal extension of the piston rod, wherein the displacement path is curved or arcuate, and wherein the actuating member comprises a curved or an arcuate shape
characterized in that the actuating member) and the piston rod (12) are releasably engaged by means of at least one gear wheel (30) meshing with correspondingly toothed surfaces of piston rod and/or actuating member, and in
that the actuating member extends between a guiding element and the at least one gear wheel to positively guide the actuating member, wherein the at least one gear wheel engages with an inside directed toothed surface of the actuating element and wherein the guiding element engages with an outside directed surface of the actuating element.

2. The drive mechanism according to claim 1, wherein the piston rod is disposed along the main longitudinal axis of the cartridge, and wherein the piston rod is rigid.

3. The drive mechanism according claim 1, wherein the actuating member intersects a sidewall section or a proximal end section of the housing.

4. The drive mechanism according to claim 1, wherein the actuating member at least in sections comprises a toothed surface meshing with a first gear wheel and wherein the piston rod comprises a toothed surface meshing with a second gear wheel, wherein first and second gear wheels are operably engaged by means of a shaft.

5. The drive mechanism according to claim 4, wherein the at least one gear wheel is unidirectionally engaged with the shaft and/or with the second gear wheel by means of a ratchet.

6. The drive mechanism according to claim 4, wherein the shaft and/or the first gear wheel and/or the second gear wheel are slidably disposed in the housing along the shaft's longitudinal direction for mutually engaging and/or disengaging of piston rod and actuating member.

7. The drive mechanism according to claim 4, wherein at least one gear wheel and/or the shaft is engaged with a displaceable display member being adapted to indicate an engagement of piston rod and actuating member and/or being adapted to indicate an actual position of the piston rod and/or of the actuating member.

8. The drive mechanism according to claim 1, wherein the actuating member at least comprises a flexibly deformable distal end section, adapted to be bended by and/or to be guided along an inner sidewall of the housing.

9. The drive mechanism according to claim 8, wherein the actuating member comprises a proximal end section, and wherein the proximal end section is less flexibly deformable than the distal end section of the actuating member.

10. The drive mechanism according to claim 1, wherein the guiding element is displaceably arranged in a through opening of the housing.

11. The drive mechanism according to claim 1, wherein the guiding element is depressible against a reset spring force provided by the actuating member.

12. A drug delivery device for dispensing of a medicinal fluid and comprising a drive mechanism according to claim 1.
